(19) United States
(12) Patent Application Publication
(10) Pub. No.: US 2019/0247671 A1
(43) Pub. Date: Aug. 15, 2019

(54) WEARABLE CARDIOVERTER DEFIBRILLATOR LATCHING CONNECTOR

(71) Applicant: West Affum Holdings Corp., Grand Cayman (KY)

(72) Inventors: Daniel R. Piha, Bellevue, WA (US); Robert R. Buchanan, Redmond, WA (US); Dallas E. Meeker, Bothell, WA (US); Douglas K. Medema, Everett, WA (US); Quan H. Nguyen, Renton, WA (US)

(21) Appl. No.: 16/277,838

(22) Filed: Feb. 15, 2019

Related U.S. Application Data
(60) Provisional application No. 62/630,995, filed on Feb. 15, 2018.

(51) Int. Cl. A61N 1/39; A61N 1/04

(52) U.S. Cl. CPC A61N 1/3904; A61N 1/046; A61N 1/3968; A61N 1/3987; A61N 1/0472; A61N 1/3993

(57) ABSTRACT

A wearable cardioverter defibrillator (“WCD”) latching connector system includes a receptacle positioned within a WCD monitor, and a connector configured to removably engage the receptacle.

### CROSS-REFERENCE TO RELATED APPLICATION

This application claims priority to and the benefit of U.S. Provisional Patent Application No. 62/630,995, filed Feb. 15, 2018, which is incorporated herein by reference in its entirety for all purposes.

### SUMMARY

The present disclosure generally relates to wearable cardioverter defibrillator (“WCD”) systems. WCD systems have become a standard of care for patients who may be candidates for a future implantable defibrillator but do not currently meet the criteria for such a device. WCDs act as a bridge between an event such as a myocardial infarction or ex-plantation of an implantable cardioverter defibrillator (“ICD”) and when the patient is a viable candidate for a new implant. WCD systems may monitor the patient's electrocardiography (“ECG”) signals twenty-four hours a day, continuously processing them to determine if defibrillation therapy is needed. WCD systems often include monitors that contain elements of the WCD system (e.g., electronics) and facilitate a patient's (or other user's) understanding of how the WCD system is operating. WCD systems may include connector systems that electrically and physically connect different components. For example, a connector system may electrically and physically connect a WCD monitor to an electrode for attachment to a patient.

In an embodiment, the present disclosure provides a WCD latching connector system for incorporation into a WCD system, the WCD latching connector system providing a shielded connection and incorporating water seals to resist water ingress. The WCD latching system has a receptacle positioned within a WCD monitor and extending through an outer wall of the WCD monitor, and a connector configured to removably engage the receptacle.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings and appendix, wherein:

FIG. 1 is a perspective view of a Wearable Cardioverter Defibrillator (“WCD”) latching connector system of the present disclosure;

FIG. 2 is an exploded partial perspective view of the WCD latching connector system of FIG. 1;

FIG. 3 is a perspective view of an aspect of the WCD latching connector system of FIG. 1;

FIG. 4 is a partial section view of the WCD latching connector system of FIG. 1;

FIG. 5 is a first perspective view of a WCD connector of the WCD latching connector system of FIG. 1;

FIG. 6 is a second perspective view of the WCD connector of FIG. 5;

FIG. 7 is a third perspective view of the WCD connector of FIG. 5;

FIG. 8 is a fourth perspective view of the WCD connector of FIG. 5;

FIG. 9 is a front view of the WCD connector of FIG. 5;

FIG. 10 is a rear view of the WCD connector of FIG. 5;

FIG. 11 is a top view of the WCD connector of FIG. 5;

FIG. 12 is a bottom view of the WCD connector of FIG. 5;

FIG. 13 is a right view of the WCD connector of FIG. 5;

FIG. 14 is a left view of the WCD connector of FIG. 5; and

FIG. 15 is a partial section view of the WCD connector of FIG. 5.

FIG. 16 is a first perspective view of another WCD connector of a WCD latching connector system of the present disclosure;

FIG. 17 is a second perspective view of the WCD connector of FIG. 16;

FIG. 18 is a third perspective view of the WCD connector of FIG. 16;

FIG. 19 is a fourth perspective view of the WCD connector of FIG. 16;

FIG. 20 is a front view of the WCD connector of FIG. 16;

FIG. 21 is a rear view of the WCD connector of FIG. 16;

FIG. 22 is a top view of the WCD connector of FIG. 16;

FIG. 23 is a bottom view of the WCD connector of FIG. 16;

FIG. 24 is a right view of the WCD connector of FIG. 16;

FIG. 25 is a left view of the WCD connector of FIG. 16; and

FIG. 26 is a partial section view of an aspect of the WCD connector of FIG. 16.

### DETAILED DESCRIPTION

The detailed description set forth below in connection with the appended drawings is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the claimed subject matter to the precise forms disclosed.

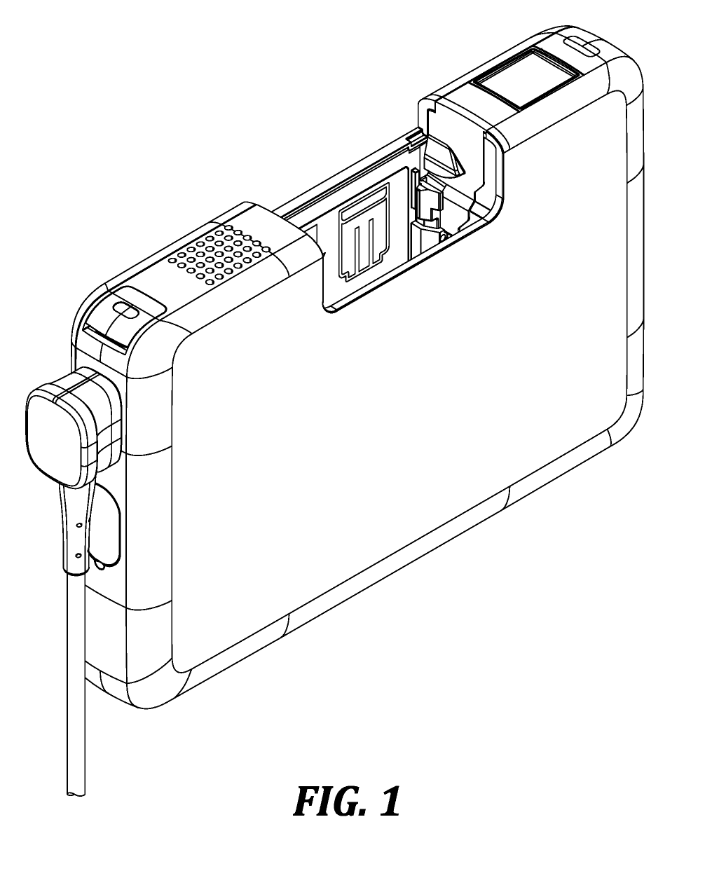

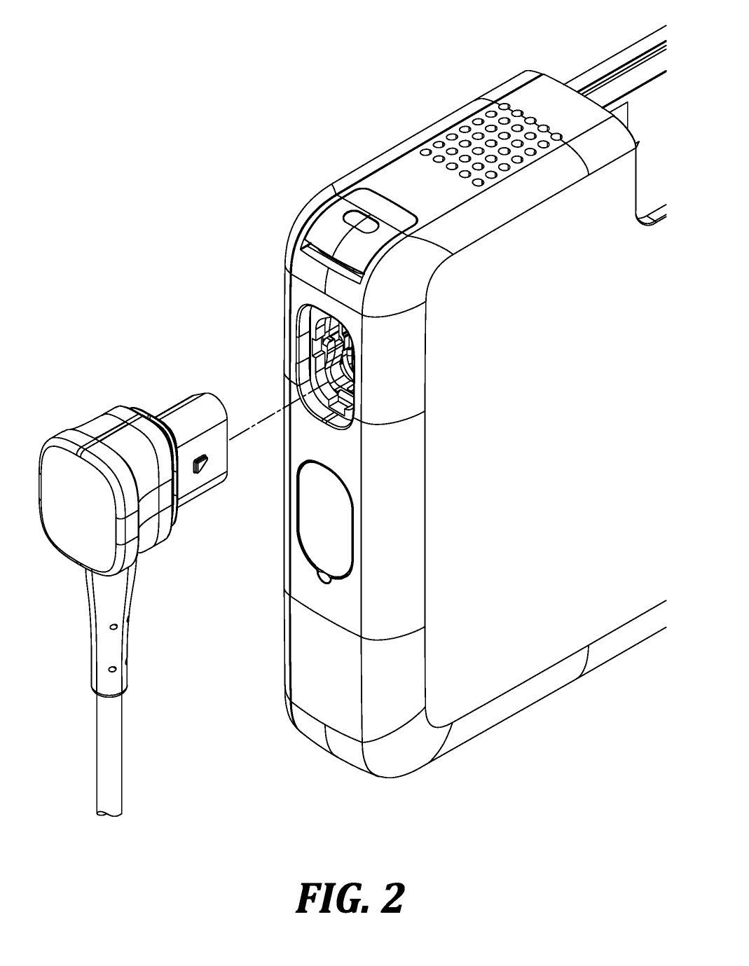

Referring to FIGS. 1-2, a wearable cardioverter defibrillator (“WCD”) system includes a WCD latching connector system that is partially integrated with a WCD monitor. The WCD system may include additional elements, including a plurality of electrodes configured for connection to a patient and to the WCD monitor, and a harness or vest for positioning the electrodes on the body of the patient.

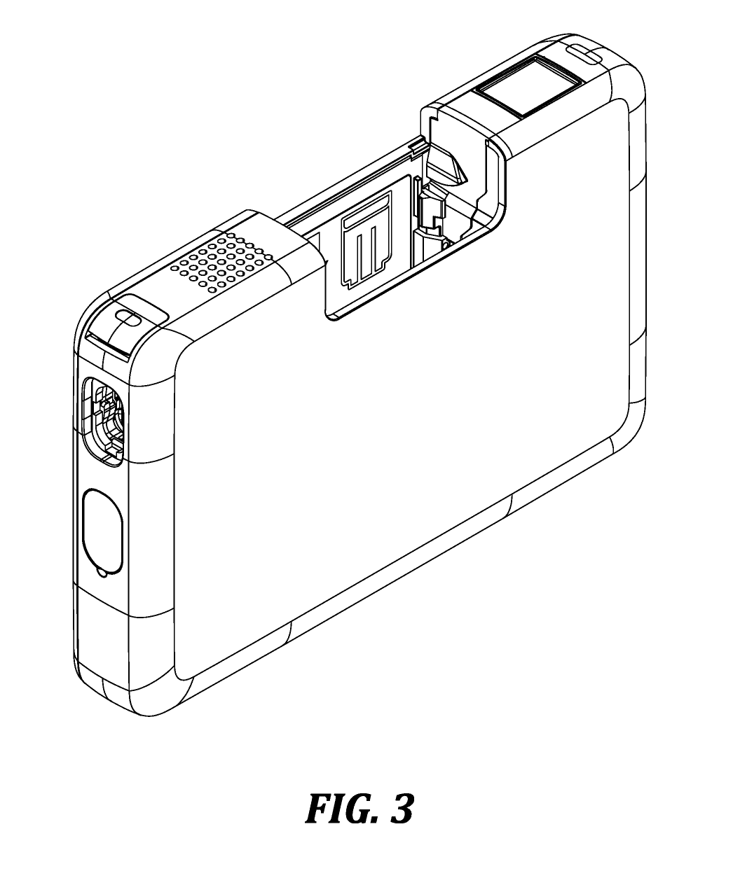

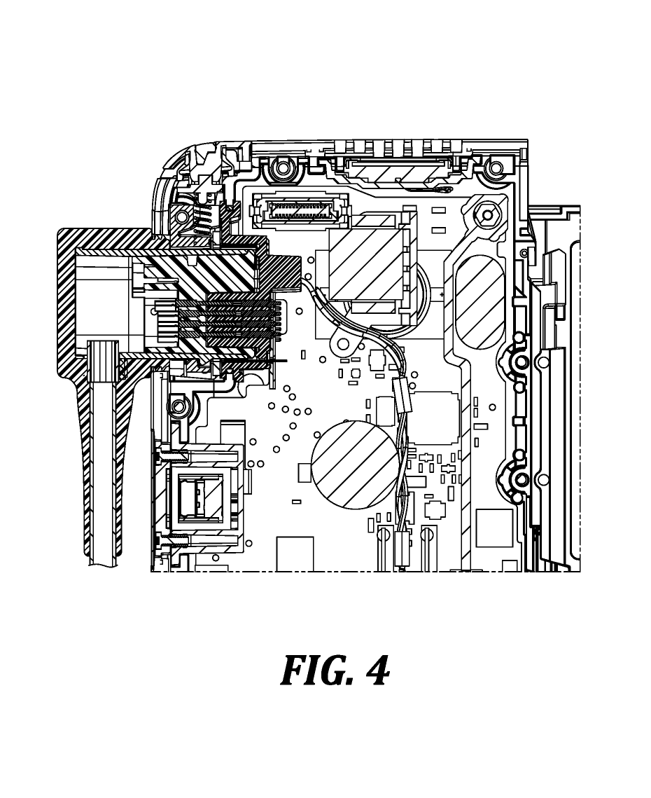

The WCD monitor shown in FIG. 3 includes an enclosure assembly that is configured to protect defibrillator electronics contained therein (electronics are partially shown in FIG. 4). The enclosure assembly may be a multi-piece assembly that includes a housing, a front cover, and a rear cover. The housing generally lies in between the front cover and the rear cover. The housing, the front cover, and the rear cover may each be constructed from one or more durable materials capable of withstanding shocks, abrasions, and other forces likely to be encountered when the WCD monitor is worn by a patient. In the non-limiting embodiment of FIGS. 1-4, the enclosure assembly is primarily constructed of one or more plastics, for example acrylonitrile butadiene styrene and/or polycarbonate.

Referring again to FIG. 2, the WCD latching connector system includes a connector and a receptacle configured to form a shielded physical and electrical connection therebetween. The connector has one or more latches, a shield (such as a metal shield), and a connector overmold. The receptacle extends through an outer wall of the WCD monitor, and includes a chamber having one or more electrical connections. The receptacle houses a shield (such as a metal shield) and a latch configured to engage the one or more latches on the connector.

In use, the WCD latching connector system may electrically and physically connect, for example, patient-facing electrodes and the WCD monitor. Other applications are contemplated. The WCD latching connector system incorporates water seals to resist ingress of water. The connector may be removably inserted into the receptacle, where it engages the WCD monitor as shown in the partial section view of FIG. 4. When the connector is inserted into the receptacle, a latch of the receptacle engages a latch of the connector, locking the connector to the receptacle. To remove the connector, a button on the WCD monitor is depressed, thereby unlatching the connector from the receptacle and enabling removal of the connector from the connector.

In an embodiment, the button includes a membrane (e.g., an elastomeric membrane) to prevent water from entering the receptacle, and in particular a chamber within the receptacle. In an embodiment, the WCD latching connector system includes one or more a high voltage (HV) connections for defibrillation therapy along with one or more low voltage connections. In an embodiment, the WCD latching connector system includes a mating shield connection in the connector and receptacle that ensures the LV and HV signals remain intact in electrically noisy environments. In an embodiment, the WCD latching connector system includes a latching system as described above that locks the connector to the receptacle and requires a button to be depressed in order to unlatch the connector to remove the connector. An embodiment may have any single or combination of the above features. In an embodiment, the WCD latching connector system includes a connector overmold to create a water seal on the WCD housing system when inserted into the receptacle to further resist liquid from entering the receptacle chamber.

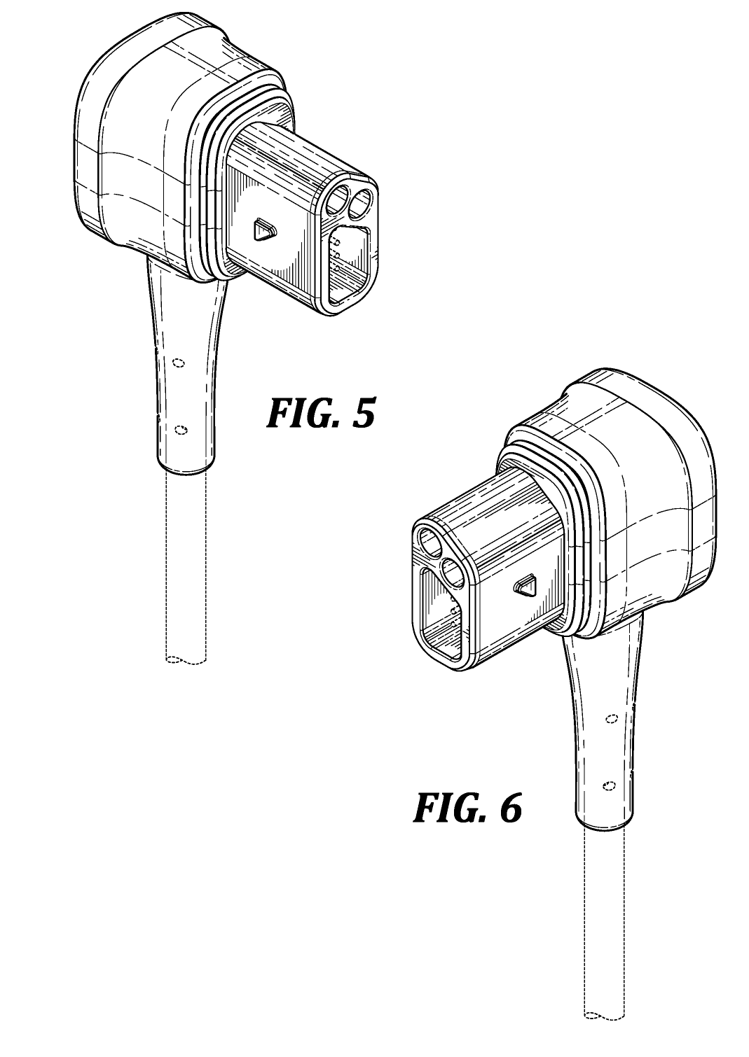

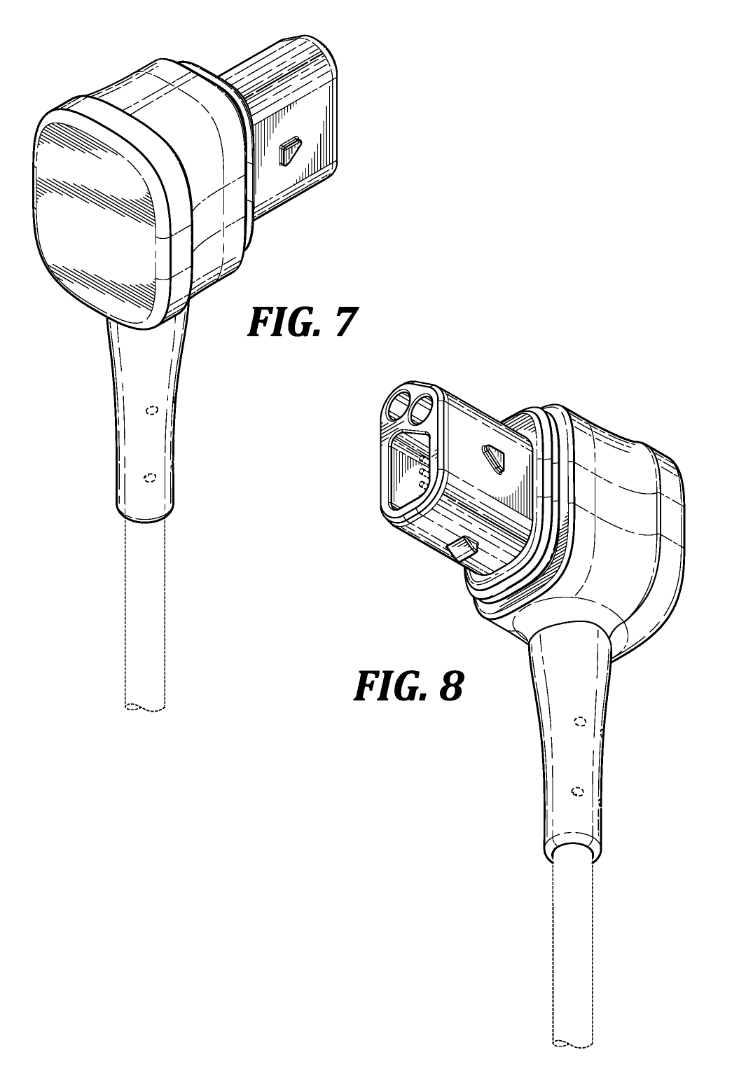

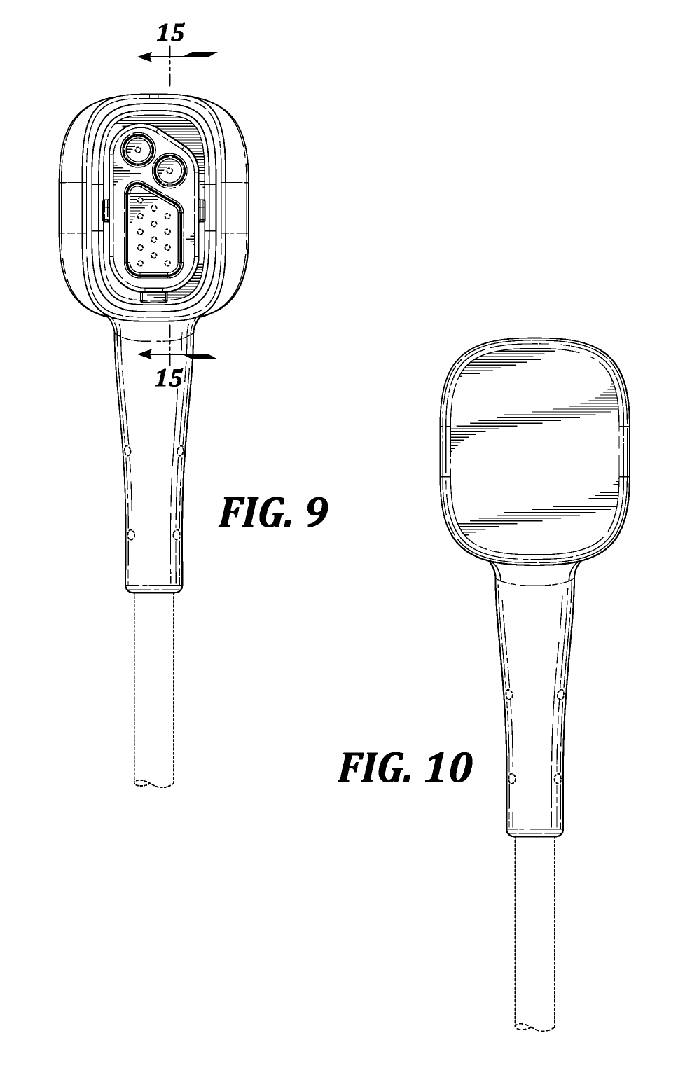

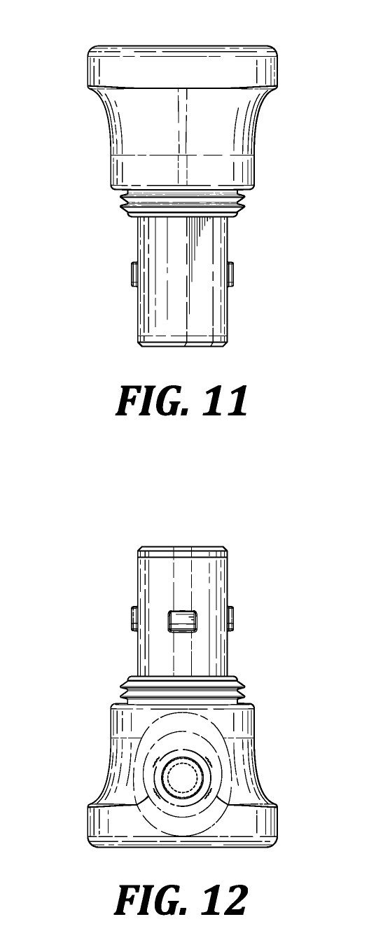

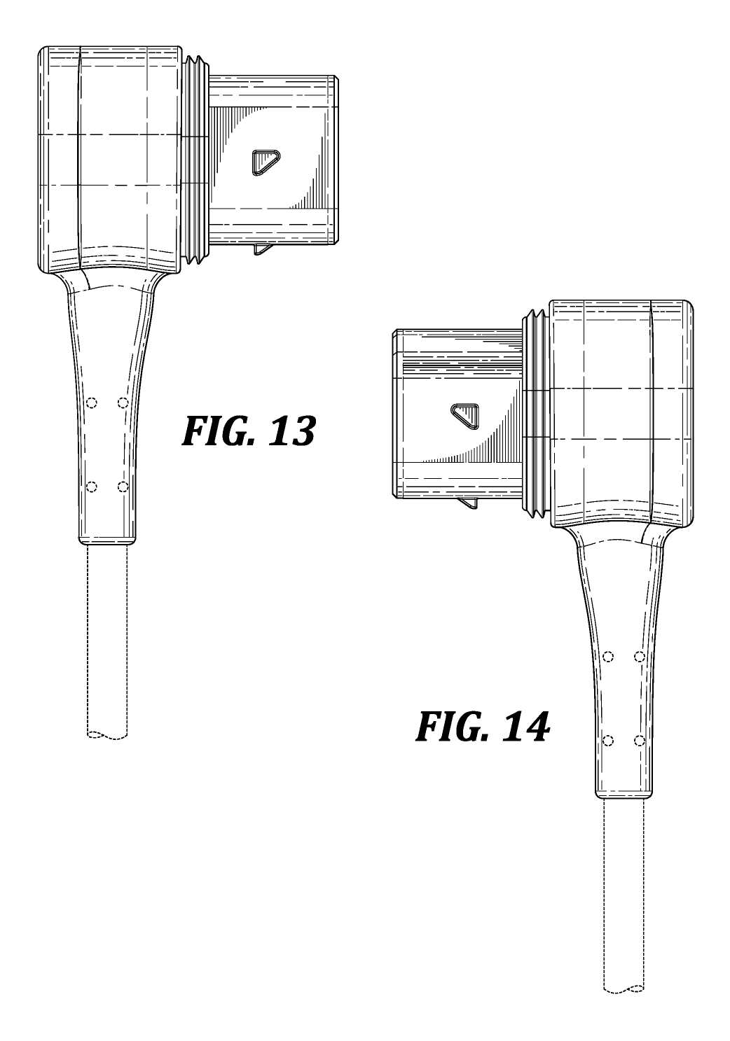

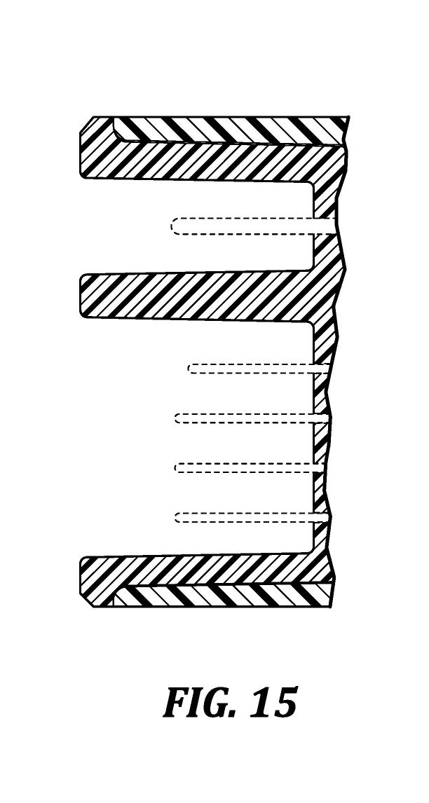

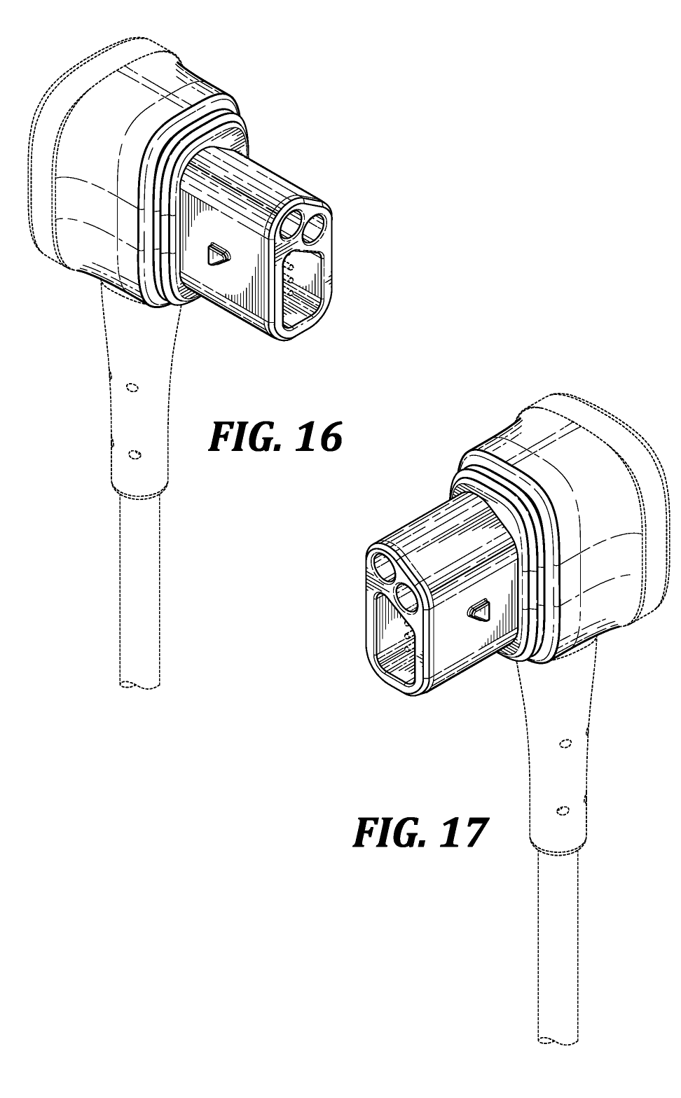

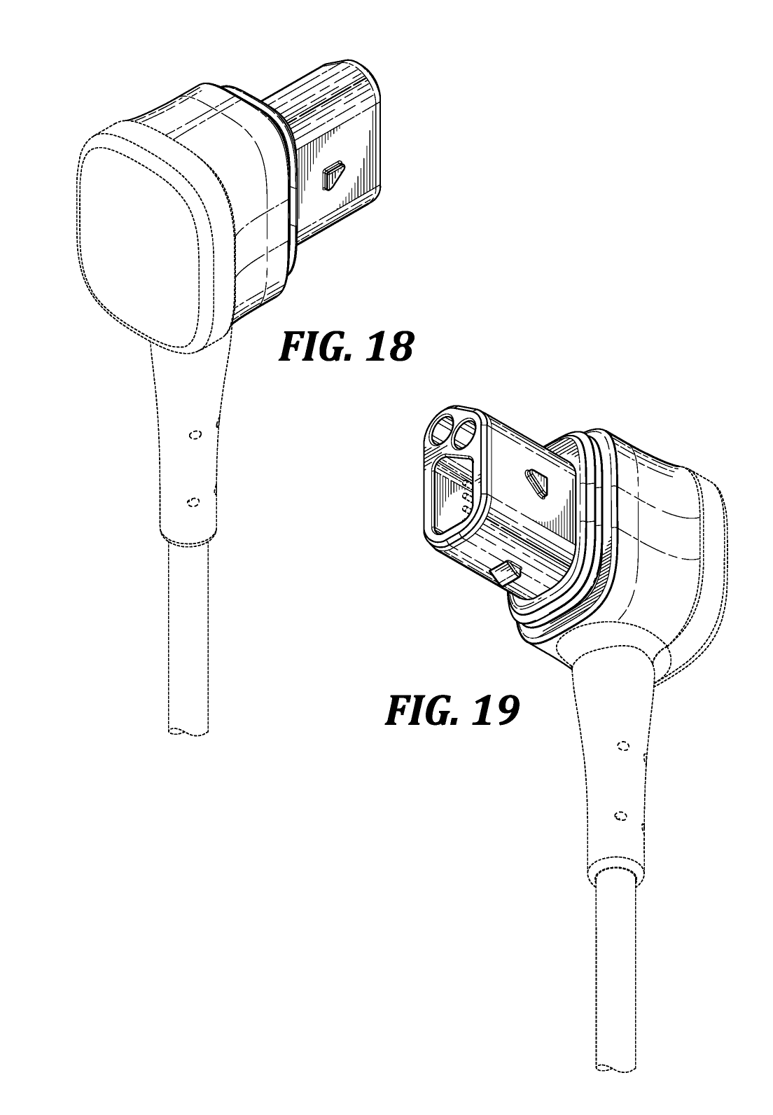

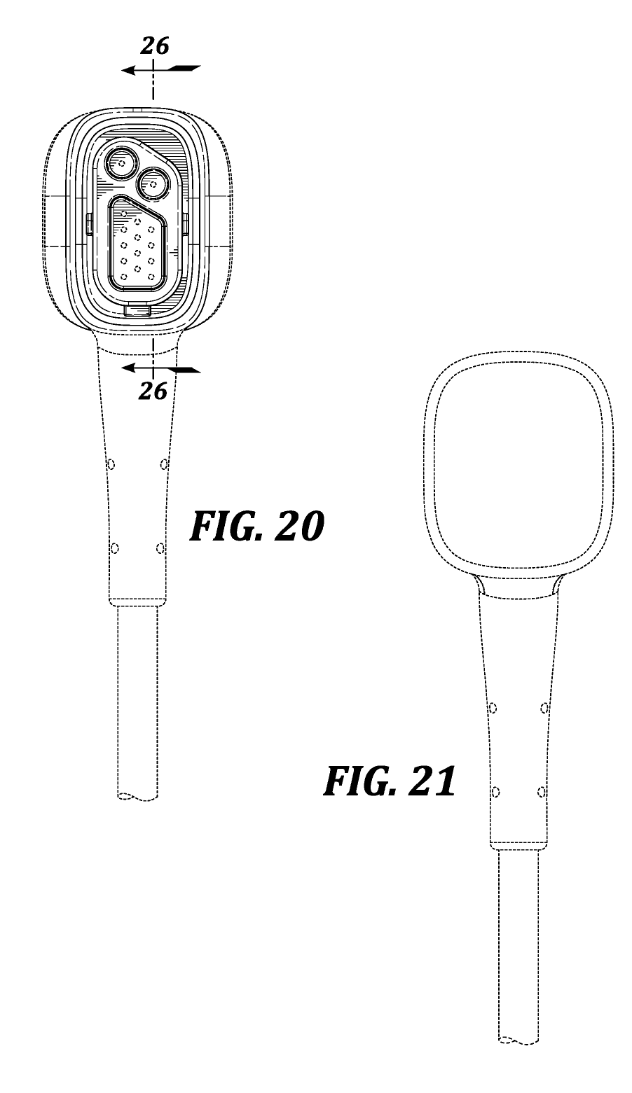

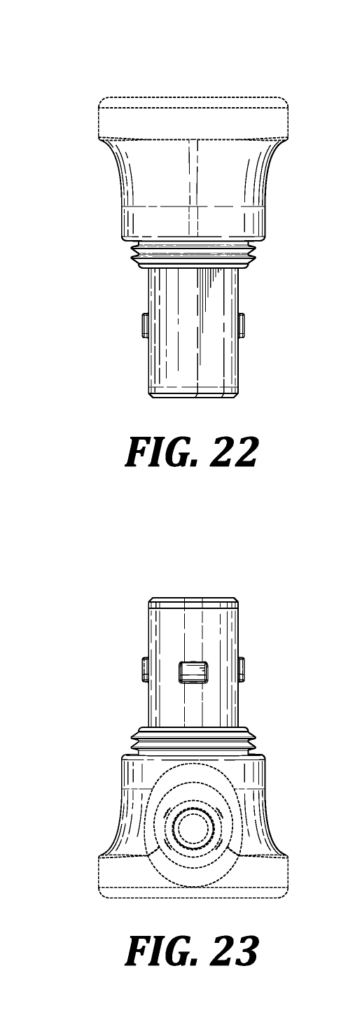

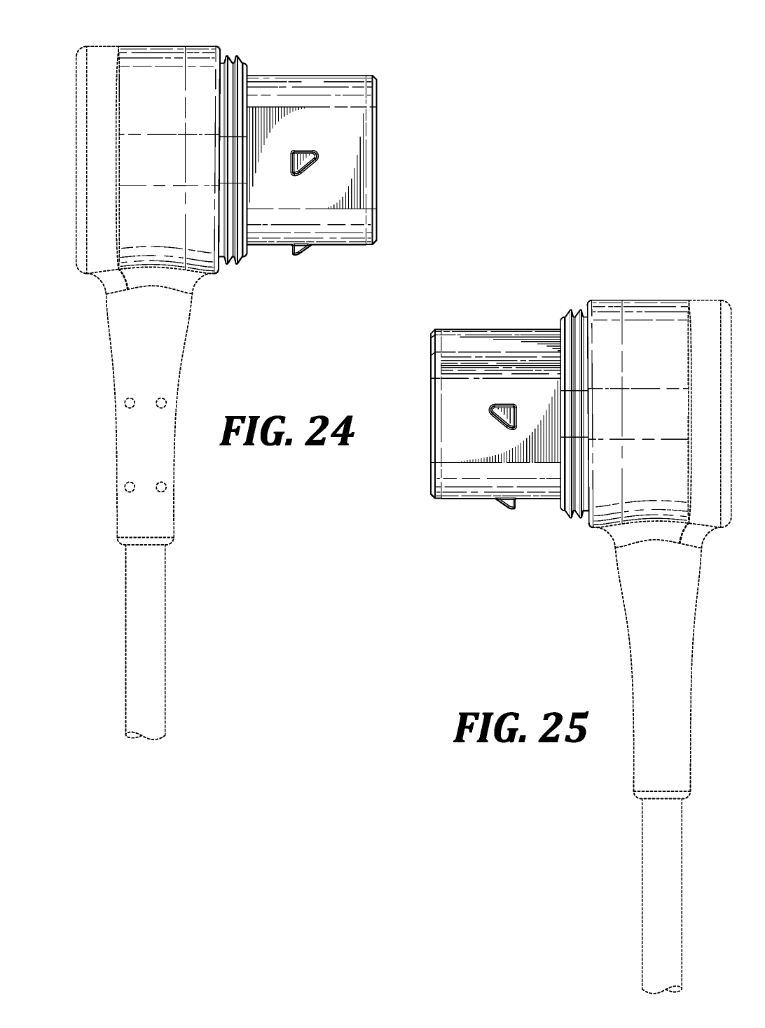

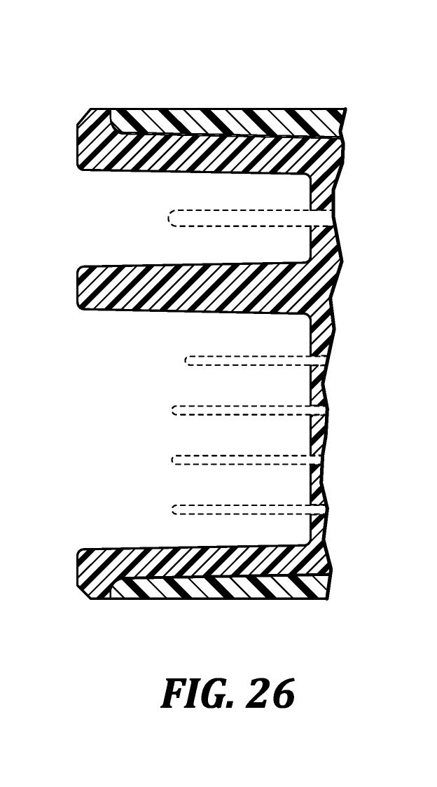

FIGS. 5-15 generally show an exemplary connector of the WCD latching connector system of FIGS. 1-4. FIGS. 16-26 generally show another exemplary connector of a WCD latching connector system of the present disclosure.

Advantages of the disclosed WCD latches connector system include actively locking the connector into the receptacle of the WCD monitor, electrically shielding the LV and HV connections to improve signal quality, and sealing the receptacle chamber from water ingress.

Embodiments of the WCD latching connector system may include combinations and sub-combinations of features described or shown in the drawings herein, including for example, embodiments that are equivalent to: providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or monitor, and/or the steps, acts, or modalities of a method.

While illustrative embodiments have been illustrated and described, it will be appreciated that various changes can be made therein without departing from the spirit and scope of this disclosure. The present application may reference directions, e.g., top, bottom, front, back, left, and right. These references are intended only to aid in understanding of the embodiments, and do not limit the orientation, location, position, of any feature of the embodiments, or otherwise limit the scope of the present disclosure.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

What is claimed is:

1. A wearable cardioverter defibrillator latching connector system, comprising:
a receptacle positioned within a wearable cardioverter defibrillator monitor and extending through an outer wall thereof; and
a connector configured to removably engage the receptacle.
